(19) United States
(12) Patent Application Publication
(10) Pub. No.: US 2026/0199566 A1
(43) Pub. Date: Jul. 16, 2026

(54) Breast Pump

(71) Applicant: MEDELA HOLDING AG, Baar (CH)

(72) Inventors: Carsten Faltum, Vitznau (CH); David Y. Cho, Arlington Heights, IL (US)

(21) Appl. No.: 19/135,442

(22) PCT Filed: Dec. 5, 2022
(86) PCT No.: PCT/IB2022/000740

§ 371 Date: Jun. 4, 2025

(51) Int. Cl. A61M 1/06

(52) U.S. Cl. CPC A61M 1/064; A61M 1/067; A61M 2205/8206; A61M 2209/088

(57) ABSTRACT

Breast pump including a breast shield that receives a breast of a lactating user, an aggregate for generating suction, a milk container providing a reservoir and a controller. For reducing weight to be borne by the bra and heat irritating the comfort level of the user the breast shield and the milk container are adapted to be joined to form an in-bra breast pump unit adapted to at least in part fit inside a bra. A power source for energizing the aggregate is provided in a power housing, not mechanically linked with the breast pump unit. A power path is provided between the aggregate and the power source. Breast pump set including a first in-bra breast pump unit and a second in-bra breast pump unit, each units fitting inside a bra and having a breast shield receiving a breast of a lactating user, and a milk container providing a reservoir for milk expressed from the breast. The first second in-bra breast pump unit includes an aggregate for generating suction for expressing the milk from the breast. The second in-bra breast pump unit includes a power source. A power path and a suction line are provided between the first and the second in-bra breast pump units.

The present invention relates to a breast pump and in particular to an in-bra breast pump which can be used to express milk from the breast of a lactating user without need to support the unit being in direct contact with the breast.

Such in-bra pump is also called a hands free pump as the mother is not forced to manually assist the breast pump during operation.

WO 2018/229 504 A1 proposes an in-bra breast pump having a housing receiving an aggregate, a control and a power source for energizing the aggregate and the control. A breast shield and a milk container are connected to the housing. In other words, all components of the breast pump are connectable to form an in-bra breast pump unit, which can be suspended by the bra of the user.

The prior art breast pump has the disadvantage of being fairly heavy and thus uncomfortable for the user. Moreover, the power source usually generates a certain amount of heat which is likewise discomfortable for the user of the breast pump.

The present invention wishes to provide a proposal to at least in part overcome the above disadvantages.

As a solution to this object, the present invention provides a breast pump including a breast shield adapted to at least in part receive a breast of a lactating user. As known in prior art, the breast pump has a milk container providing a reservoir for receiving expressed milk. The milk is expressed by suction generated by an aggregate. According to the inventive concept, the breast shield and the milk container are adapted to be joined to form an in-bra breast pump unit, which in-bra breast pump unit is adapted to at least in part fit inside a bra of the user. This means that the in-bra breast pump unit of the present invention is designed to be suspended and thus held by the bra of the user. The dimensions of the in-bra breast pump unit may be partially larger than the textile material forming the bra. In other words, the in-bra breast pump unit may project beyond the contour of the bra. Nevertheless, the in-bra breast pump unit is designed to receive at least a part of the breast of a lactating user, in particular the nipple of such breast, without need to support the in-bra breast pump unit other than with the bra.

According to the concept of the present invention, the power source of the breast pump is provided in a power housing, which is operationally linked with the in-bra breast pump unit but is not mechanically coupled with the in-bra breast pump unit comprising the aggregate. A power unit housing the power source may be placed on any surface next to the user or may be adapted to be worn by the user, e.g. by connecting to a belt or the waistband of a trouser or a skirt.

Most preferably, the power source preferable forming part of a of power unit is attached or attachable to a second in-bra breast pump unit, which does not have an aggregate. According to this aspect, which will be detailed hereinafter, all components of an aggregate driven breast pump are provided to be wearable within the bra of the user, but in two different cups of such bra. In other words, all components used for simultaneously expressing milk from the left and the right breast are wearable within the bra. At least some components e.g. for controlling the breast pump, driving the breast pump and/or interacting with the user of the breast pump may be shared and may only be provided in one of the two in-bra breast pump units or respective housings i.e. may only be worn by one of the cup of such bra.

Further, a functional entity of at least one of the components may be split with the result that a portion of the functional entity may be provided by one of the in-bra breast pump units while another portion of the functional entity may be provided by the other of the in-bra breast pump units. Each of the in-bra breast pump units will at least have one breast shield and one milk container, which are coupled, preferably detachably coupled with each other to form the core of the breast pump unit. The two breast pump units are each wearable inside a bra of the user. Each of the two breast pump units are usually adapted to be detachably coupled with at least another component. Each of the two breast pump units are usually linked via a power line, a suction line and/or data line. Via the power line electric energy for driving and/or controlling components of the pumps can be exchanged between the two breast pump units. Via the suction line negative pressure for expressing milk can be exchanged between the two breast pump units. Via the data line data for controlling components of the pumps can be exchanged between the two breast pump units. In particular the data line may be replaced by a wireless data path provided between the two breast pump units.

Thus, the costs for making a set of two breast pump units, each being wearable within the bra, is reduced and so may be the weight of each of the breast pump units, the heat generated by each of the breast pump units and/or the noise of each of the breast pump units.

The comparable heavy power source, which dissipates heat in the course of energizing the aggregate, may be placed further remote from the body of the user or can form part of the other of the in-bar breast pump units.

According to a preferred embodiment of the present invention, the in-bra breast pump unit comprises a housing receiving the aggregate. This housing is detachably coupled with said breast shield and said milk container. Coupling may be provided directly or indirectly. With a direct coupling, the housing is in direct mechanical contact with the breast shield or the milk container. An indirectly coupled housing is mechanically linked with the breast shield and the milk container without necessarily having a direct mechanical link which attaches the housing to the breast shield and/or the milk container. The housing may be received within a receptacle provided within the breast shield and/or the milk container. The housing may be arranged between the breast shield and the milk container. Most preferably, the housing will be coupled such that the housing is provided within an outer surface of the in-bra breast pump unit projecting the body of the user, which outer surface imitates the shape of a female breast. In other words, the in-bra breast pump unit shall preferably be designed as known in prior art with a generally conical contact surface for the breast provided by the breast shield, which breast shield has a nipple tunnel for receiving the nipple, which breast shield may at least in part be received within or project into the milk container. The housing may be arranged above or below the milk container, which orientation refers to the general use condition of the breast pump in which the nipple tunnel extends essentially horizontally, i.e. the user is assumed to have a position in which at least the torso is upright, i.e. vertically extended (standing or sitting). The outer surfaces projecting from the body of the user shall be provided without step to generate a smooth outer surface projecting from the body of the user. The outer surfaces of the in-bra breast pump unit other than the conical contact surface may be conical or flat and conical.

A controller may be provided within the housing or the power housing. The later provides the further advantage that heat generated by the controller during control of the aggregate will not reach the body of the user or will not sum up to the heat generated by the aggregate, in particular the breast. The breast of a lactating user is in particular sensible.

The controller as a functional entity may have a portion for controlling the aggregate and a portion providing a user interface .. The user interface may be provided with a light source to illuminate buttons of the user interface. The user interface may as well have optical indicators allowing the user to obtain information on the operational status of the breast pump. Respective information may include fill level information giving information on the volume of expressed milk received within the reservoir. The illumination means mentioned above will allow the user to observe such information in the dark and/or allow swift and secure identification of different elements or functions of the user interface.

The interface may be split between the housing and the power housing. In other words, some portions of the interface may be provided in the power housing while other portions of the interface in particular those assigned to alter operational parameters of the aggregate may be provided within the housing. The interface of the power housing will at least comprise an on/off switch to activate the breast pump and to open the power path to transmit electric energy from the power source to the aggregate.

The aggregate may comprise an electric motor, which e.g. can be driven by a Piezo drive or a magnetic drive. For generating a negative pressure, the aggregate may comprise or be connected to a membrane or a piston. In general, the entire source of a negative pressure generating suction for expressing milk is located within the housing. The realization of the aggregate by a piezo drive pump as described in general in WO 2018/229 504 A1 or US 2017/095599A1 is in particular advantageous due to the limited weight of such aggregate.

A membrane and/or piston driven by the electric motor may be provided at least in part within the milk container and/or within the breast shield, preferably at an interface between the housing and at least one of the milk container and/or within the breast shield. Specifically, the membrane may be connected to and/or received within the milk container and/or the breast shield.

Specifically, the membrane may be attached to the milk container and/or the breast shield and/or the housing to separate a fluid side, which fluid side is or can be wetted by the expressed milk, from a working side of a pump arrangement. The working side may be in contact with a free end of a piston activated by the motor or may be activated by a working fluid like air. The working fluid may be cyclically driven by the electric motor to generate a cyclic suction profile within the nipple tunnel. The above mentioned membrane in particular closes off a fluid system within the breast shield which is in fluid communication with a nipple tunnel of the breast shield receiving the nipple of a lactating user. Thus, the aggregate is separated from the milk side by the membrane.

According to a preferred embodiment of the present invention, a power line is coupled to said aggregate housing and said in-bra breast pump unit. In other words, such power line extends between the aggregate housing and the in-bra breast pump unit. Respective power line may preferably be coupled to the housing. Alternatively or additionally, a suction line may be coupled to said aggregate housing and said in-bra breast pump unit.

The power line will allow transmittal of electric energy between the power source and the aggregate and/or the control. Further, physical lines may be provided between the aggregate housing and the in-bra breast pump unit, e.g. to exchange data between those two separate items of the breast pump.

Data and/or energy may likewise be exchanged between the aggregate housing and the in-bra breast pump unit by wireless means. A wireless energy connection may be provided via power transfer modulated by control signals. Thus, the power path or a data path may not necessarily but preferably be provided by a physical wire, i.e. a power line.

In order to reduce the number of overall strings extending between the in-bra breast pump unit and the aggregate housing, the breast pump preferably has a unitary connection line which integrates the suction line and the at least one power line. Preferably, the unitary connection line comprises any line for exchanging data, fluid or energy between the in-bra breast pump unit and the aggregate housing. The unitary connection line will be the only physical connection between the two components of the breast pump.

The suction line will transmit suction from the housing to the power housing, in case the same is attached to another in-bra breast pump unit of a set of two breast pump units. Such set will have two in-bra breast pump units for simultaneous expression of milk from the left and the right breast with only one power source within the power source housing attached to one of the units and a housing receiving at least the aggregate, which housing is attached to the other of the in-bra breast pump units. In such a set, the overall weight for the aggregate and the means for energizing, controlling and possibly monitoring performance of the aggregate can be split between the two units. In such a set, the milk container and the breast shield of each in-bra breast pump unit are identical at least with respect to their mating surfaces. As usual, the breast shield may be provided with different sizes or shapes to comply with varying anatomic conditions of the user. The mating surfaces may include those surfaces adapted to hold and functionally incorporate the housing and the power housing to each of the in-bra breast pump units.

The design of the set is preferably selected such that the power housing and the housing, both equipped with their assigned means, have approximately the same weight. Both, the housing and the power housing preferably have identical interfaces for coupling with the in-bra breast pump unit in terms of mechanical and functional linking between each housing and the respective pump unit.

Further details and advantages of the present invention will become apparent from the following description of an embodiment in connection with the drawing. In the drawings:

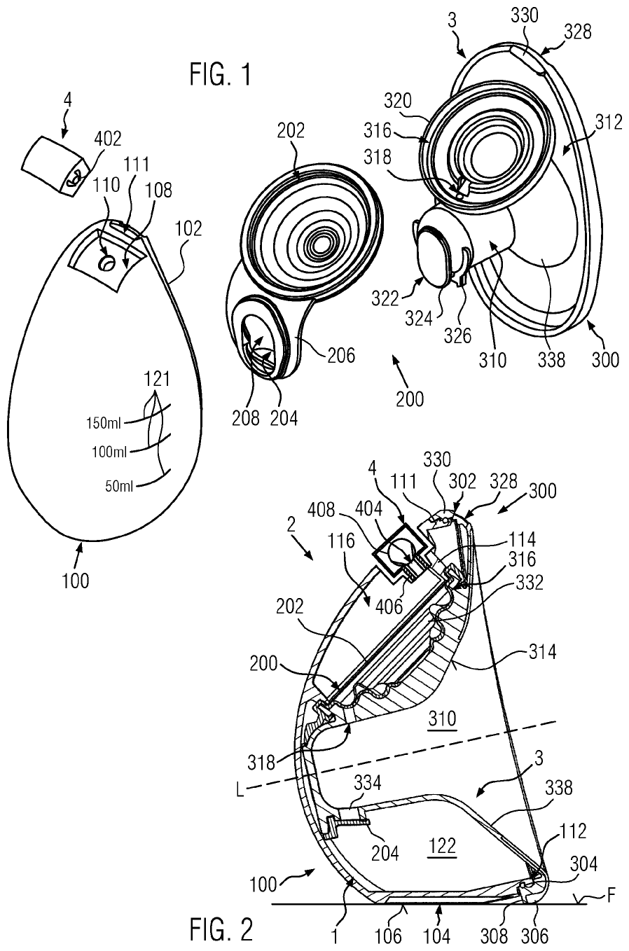

FIG. 1 is a perspective exploded view of the embodiment;

FIG. 2 is a cross-sectional view of the embodiment of FIG. 1, which cross-sectional view contains the longitudinal axis of the nipple tunnel and extends parallel to a plane comprising the vertical axis;

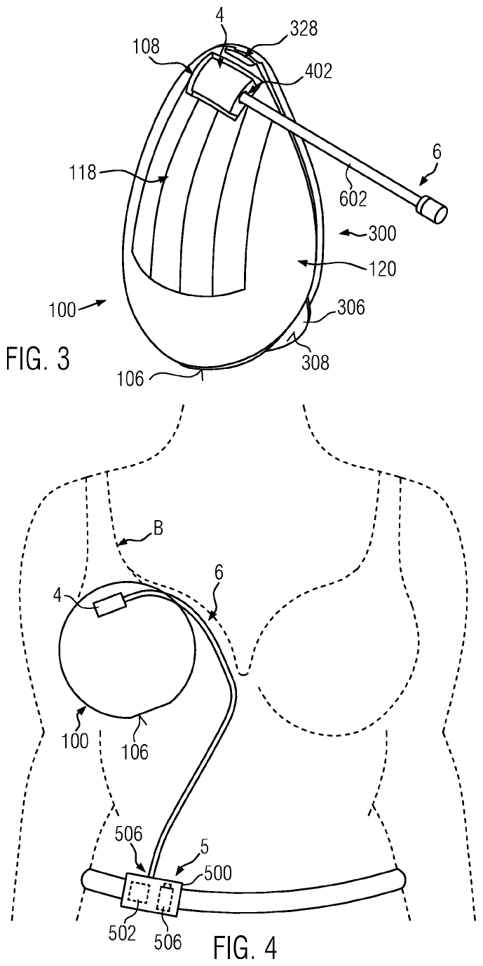

FIG. 3 is a perspective view of the front side of the embodiment of FIGS. 1 and 2;

FIG. 4 is a front view showing the embodiment of FIGS. 1 to 3 in use and

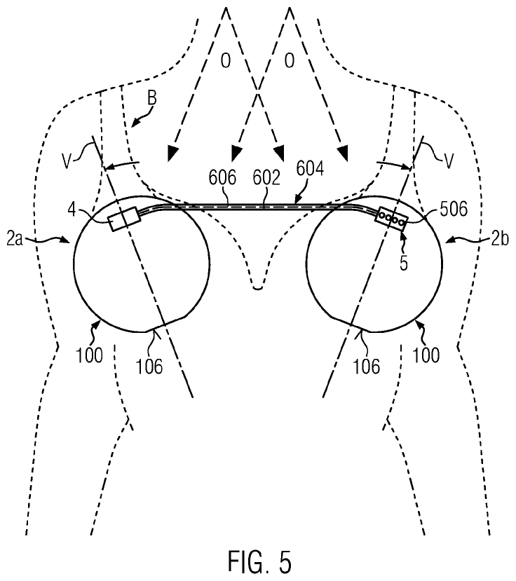

FIG. 5 is a front view of a second embodiment of the present invention in use.

In the Figures, reference numeral 100 identifies an outer shell element 100, reference numeral 200 identifies a media separation element and reference numeral 300 identifies a breast shield element providing a breast shield 3. The breast shield element 300 provides a lit cooperating with the shell element 100 to define a milk container 1 of an in-bra breast pump unit 2. Reference numeral 4 identifies a housing of the in-bra breast pump unit 3, which housing is in fluid communication and data communication with a power unit 5 shown in FIG. 4 via a power path 6. The housing 4 is detachable from the milk container 1. The power unit 5 is separate from the in-bra breast pump unit 3 and not mechanically linked with said in-bra breast pump unit 3.

The outer shell element 100 is spherical and defines a plane interface 102 surrounded by a circumferential rim 302 of the breast shield element 300. A sealing arrangement in form of a circumferential seal 304 is provided between the interface 102 and the circumferential rim 302. At a bottom of the in-bra breast pump unit 2, the breast shield element 300 has a wing 306 defining a detachment surface 308. Adjacent to said detachment surface 308, the outer shell element 100 provides a flat recess 104 as an interruption to the spherical outer surface of the outer shell element 100, which recess 104 provides a standing surface 106. For opening the embodiment as depicted in FIG. 2, the user will grasp around the outer shell element 100 such that the thumb of the hand grasping around the outer shell element 100 will lay against the holding surface 106. The thumb of the other hand will cooperate with the detachment surface 308 while the fingers of the hand will at least in part cooperate with the circumferential rim 302 at the top of the pump to remove the breast shield element 300 from the outer shell element 100.

Near the top, the outer shell element 100 provides a receptacle 108 formed as a recess within the spherical outer surface and adapted to receive the housing 4. The housing 4 i.e. a circumference thereof is provided with a power interface 402, which is connected with the unitary connection line 6 depicted in FIG. 3. The receptacle 108 has a longish extension. Due to the spherical constitution of the outer shell element 100, the opposing lateral rims surrounding the receptacle 108 are arranged lower than the axial extension of the power interface 402. Thus, the unitary connection line 6 can extend form the housing 4 in a lateral extension as shown in FIG. 3. The housing 4 has a central bore 404 extended by a protrusion 406 sealingly cooperating with a working medium inlet 110 provided in the center of the receptacle 108 (compare FIG. 2). Thus, the housing 4 can be arranged in an alignment 180° tilted around the working medium inlet to provide connection of the connection line 6 in a lateral extension opposite to the extension shown in FIG. 3.

The breast shield element 300 of the present embodiment is a co-injection molded unitary element having a nipple tunnel 310 made of a transparent material and a flange section 312 made of an opaque material, which flange section 312 provides a contact surface 314 to at least in part contact the breast of the user. Due to the transparent properties of the nipple tunnel 310, the user can confirm proper alignment of the nipple within the nipple tunnel 310. Above the nipple tunnel 310, the breast shield element 300 defines a pumping chamber housing 316 which via a pumping inlet 318 is in fluid communication with the nipple tunnel 310. The pumping chamber 316 has a flat upper rim 320. The upper rim 320 is ring-shaped.

An inner end 322 of the nipple tunnel 310 is provided with a securing interface 324. The bottom side of the nipple tunnel 310 is projected by a valve receptacle 326.

At the top of the breast shield element 300, there is provided an open spout 328, which is projected by a snapping hook 330 provided by the breast shield element 300 and cooperating with a snapping counter surface 111 provided by a ring segment 112 of the outer shell element 100 with the circumferential seal interdisposed.

The media separation element 200 has a membrane 202 and a valve 204 being connected by a connection section 206 having a securing opening 208. The membrane 202, the valve 204 and the connection section 206 are realized in a single unitary element i.e. the media separation element 200.

As visible from FIG. 2, the outer shell element 100 has a circumferential rim 114 projecting inwardly from the concave inner surface of the outer shell element 100.

For assembly, the media separation element 200 will be secured to the inner end 322 of the nipple tunnel 310 by inserting the securing interface 324 at least in part into the securing opening 208. Mating connection surfaces of the media separation element 200 and the securing interface 324 will provide attachment of the two elements 100, 200. Thereafter, the membrane 202 will be tilted and secured against the upper rim 320 to close a pumping chamber 332. As a result and as visible from FIG. 2, the membrane 202 grasps around the upper rim 320 for proper attachment of the membrane 202 against the breast shield element 300, specifically the pumping chamber housing 316. On the other side, the valve 204 will be tilted to be connected within the valve receptacle 326 and abut against a milk outlet 334 provided on an outer circumference of the nipple tunnel 310.

In the further course of the assembly, the preassembled group comprising the media separation element 200 and the breast shield element 300 is inserted into the outer shell element 100. In the course of this assembly, the circumferential rim 114 will abut the membrane 202 so that the membrane 202 is sealed between the breast shield element 300 and the outer shell element 100.

Thus, a working fluid chamber 116 is provided within the outer shell element 100, which working fluid chamber 116 is in communication with an aggregate 408 received in the housing 4. The aggregate 408 is a pump which provides a cyclic air flow into and out of the working fluid chamber 116 via the working medium inlet 110.

FIG. 3 elucidates formation of the outer shell element 100 of two plastic materials with different transparent properties. For the following discussion, referral is made to a longitudinal axis of the nipple tunnel identified with reference numeral L in FIGS. 2 and 3 and a vertical axis V extending perpendicular to the longitudinal axis L and extending in the same plane, which vertical axis defines the sectional view of FIG. 2. The embodiment is symmetrical with respect to the vertical axis V.

The same applies to the different sections of the outer shell element 100. A first section 118 covers the nipple tunnel 310 and, thus, is arranged in a central portion of the outer shell element 100. The first section 118 is made of a frosted transparent thermoplastic material. A second section 120 is provided laterally to the first section 118 and is made of slightly frosted transparent plastic material having a higher degree of transparency than the first section 118. The two sections 118, 120 are joined by co-injection molding. The lateral sides of the second section 120 are each provided with scale-lines 121 identifying the amount of milk collected within the pump. Alternatively scale-lines may be provided on the breast shield 4. In the present embodiment, the second section 120 is U-shaped and also extends over a central area of the outer shell element 100. In the present embodiment, the second section 120 provides the bottom of the outer shell element 100.

FIG. 1 exemplifies the breast shield element 300 being made of two different materials. The nipple tunnel 310 as well as a conical section 338 emerging from the nipple tunnel 310 are made of a clear transparent material, while the flange section 312 is made of a frosted or opaque plastic material. This constitution allows the user to check nipple alignment within the nipple tunnel 310 through the breast shield element 300.

In use, which is shown in FIG. or 5, the breast pump is arranged with the vertical axis V slightly inclined outwardly below the breast pump. Thus, a view O of the user will be directed to the lateral sides of the outer shell element and, thus, the second section which will allow the user to observe milk flow and nipple alignment as well as e.g. valve operation.

The power unit 5 shown in FIG. 4 has a power housing 500, which houses a controller 502 and a power source 504. The power housing 500 is provided with a user interface 506 for altering and/or indicating operational parameters of the aggregate 408. In the example, the power path 6 is provided by a power line 602, which is a physical current path for supplying the aggregate 408 with electric energy from the power source 506 in form a battery. The user interface 506 may comprise at least one element like a button to change operational conditions of the aggregate 408. The user interface 506 may comprise at least one optical indicator for indicating an operational condition e.g. of the power source 506, the controller 502 or the fill level of the reservoir 122.

The housing 4 may be equipped with a fill sensor or at least an interface of such fill sensor. Such fill sensor may be connected with the housing 4 and/or the controller 502 by a data connection (physical or Bluetooth-not shown). In case of a physical connection with the controller 502, the power line 602 and the data connection may be provided by a common connection line 504.

In the embodiment, the power line 602 is fixedly attached with one end to the housing 4 and with the other and to the power housing 500. For left and right breast use of the pump the power line 602 can be directed towards the center of the body thanks to the two different orientations in which the housing 4 is rotated by 180° within the receptacle 108. The outer shell 100 is avocado-shaped. The outer shell element 100 has the largest width approximately at level of the longitudinal axis L, the width being an extension perpendicular to the plane containing the vertical axis and the longitudinal axis. The outer shell element 100 and thus the breast shield element have the largest extension in height direction in the plane containing the vertical axis V. The longitudinal axis L is provided below the middle height of the outer shell element 100, the height being the extension parallel to the vertical axis. Due to this, the center of gravity of the in-bra breast pump unit 2 is essentially leveled with the longitudinal axis or slightly below the longitudinal axis L. Due to this center of gravity, the pump unit 2 is inclined to assume a positon in which the open spout 328 assumes the highest point of the pump, whereas the holding surface 106 assumes the lowest point. In fact and as evident from FIG. 2, this standing surface 106 securely supports an upright position of a pump being placed on a flat horizontal surface F.

Due to the avocado shape of the outer shell element 100, the breast pump of the embodiment can be stored at least in part within a bra B—FIG. 4. Thus, the in-bra breast pump unit 2 is a hands-free pump.

In operation, the aggregate 408 cyclically evacuates the working fluid chamber 116, which causes the membrane 202 to expand either towards the working fluid chamber 116 or towards the pumping chamber 332.

Through the pumping inlet 318, the pressure profile thus generated in the pumping chamber 332 is transferred into the nipple tunnel 310. Due to this, milk is expressed, which milk is lead down into the reservoir 122 sandwiched between the outer shell element 100 and the breast shield element 300. The reservoir 122 in fact assumes the entire inner space within the outer shell element 100 which is not covered by material of the breast shield element 300 or the media separation element 200 or the volume of the working fluid chamber 116 and the pumping chamber 332. The reservoir 122 is in communication with the atmosphere through the open spout 328. As the open spout 328 is arranged at the top of the in-bra breast pump unit 2 and as the open spout 328 has a rather small dimension, spillage of milk expressed and contained within the reservoir 122 can be avoided.

After expressing milk, the pump will be taken out of the bra B and can rest on the standing surface 106 in an upright position. The expressed milk can be poured out through the open spout 328 by rotating the pump upside down. During pumping operation and filling up the reservoir 122 with milk, air can vent through the open spout 328.

In the present embodiment, an outer surface of the breast pump unit 2 is provided essentially by the outer shell element 100 and the breast shield element 300 only. The breast shield element 300 provides the breast shield 3 adapted to be laid against the beast of the user, specifically by means of the contact surface 314.

The outer shell element 100 and the breast shield element 300 are connected with each other. The outer periphery of breast shield element 300 is level with the outer periphery of the outer shell element 100. Free outer edges formed by the outer periphery of the breast shield element 300 and/or the outer shell element 100 are avoided in favor of a smooth outer housing 2 of the breast pump. The outer periphery of the outer shell element 100 is grasped around by and received within the outer periphery of breast shield element 300.

The working fluid chamber 116, the pumping chamber 332, and the milk compartment 122 are exclusively defined by the three individual but unitary elements discussed in detail above, i.e. the outer shell element 100, the media separation element 200, and the breast shield element 300. As the media separation element 200 as a single unitary element provides the membrane 202 as well as the valve 204, the breast pump of the present invention can be manufactured with a reduced number of parts. The media separation element 200 can easily be mounted on the breast shield element 300. Assembly of the in-bra breast pump unit 2 is facilitated due to the reduced number of individual parts to be handled as well as the possibility to temporarily attach the media separation element 200 against the inner end 322 of the nipple tunnel 310. Due to the cooperating surfaces provided by the circumferential rim 114 and the upper rim 320, the membrane 202 is securely sandwiched to seal the fluid working chamber 116 and the pumping chamber 332 after assembly of the pump.

Transparent or slightly frosted sections of the breast pump unit 2 allow visibility through the outer shell element 100 and at least portions of the breast shield element 300, in particular the nipple tunnel 310 to observe nipple alignment and/or milk flow and seize thereof indication termination of milk flow.

In the embodiment of FIG. 5, the power unit 5 has a power housing 500 being shaped as the housing 4. Thus, the power housing 500 can be arranged within the receptacle 108. As in the previous embodiment, the power housing 500 houses the controller 502 and the power source 505 and is provided with the user interface 506. In addition, the power housing 500 is provided with an internal fluid path connecting an interface 508 with the bore and protrusion arrangement 404, 406 as described before. In FIG. 5, reference numeral 604 identifies a common connection line, which incorporates the power line 602 and a suction line 606. Thus, electric energy can be transferred from the power housing 500 to the aggregate 408 within the housing 4 via the power line 604 and cyclic air flow can be transmitted from the housing 4 to the working fluid chamber 116 of the in-bra breast pump unit 2b shown on the right hand side of FIG. 5 via the suction line 606. The flow resistance for the airflow from the aggregate 408 into the working fluid chamber 116 of the in-bra breast pump unit 2a on the left side of FIG. 5 and of the in-bra breast pump unit 2b on the right side of FIG. 5 is adjusted to provide a hydraulic balance. Thus, each of the working fluid chambers 116 receives approximately half of the airflow generated by the aggregate 408.

### LIST OF REFERENCE SIGNS

1 milk container
2 in-bra breast pump unit
3 breast shield
4 housing
5 power unit
6 power path
100 outer shell element
102 interface
104 recess
106 standing surface
108 receptacle
110 working medium inlet
111 snapping counter surface
112 ring segment
114 circumferential rim
116 working fluid chamber
118 first section
120 second section
121 scale line
122 reservoir
200 media separation element
202 membrane
204 valve
206 connection section
208 securing opening
300 breast shield element
302 circumferential rim
304 circumferential seal
306 wing
308 detachment surface
310 nipple tunnel
312 flange section
314 contact surface
316 pumping chamber housing
318 pumping inlet
320 upper rim
322 inner end
324 securing interface
326 valve receptacle
328 open spout
330 snapping hook
332 pumping chamber
334 milk outlet
338 conical section
402 power interface
404 bore
406 protrusion
408 aggregate
500 power housing
502 controller
506 user interface
602 power line
604 connection line
606 suction line/data line
L longitudinal axis of the nipple tunnel
V vertical axis
F flat surface
B bra
O view

1-11. (canceled)

12. A breast pump including a breast shield adapted to at least in part receive a breast of a lactating user, an aggregate for generating suction for expressing milk from the breast, a milk container providing a reservoir for the expressed milk and a controller for controlling the aggregate, the breast shield and the milk container adapted to be joined to form an in-bra breast pump unit adapted to at least in part fit inside a bra, the in-bra breast pump unit comprising the aggregate and a power source for energizing the aggregate, which power source is provided in a power housing, which is not mechanically linked with said in-bra breast pump unit, a power path is provided between the aggregate and the power source, and the aggregate further comprising an additional power source.

13. The breast pump according to claim 12, the in-bra breast pump unit further comprising a housing receiving the aggregate, which housing is detachably coupled with said breast shield and said milk container.

14. The breast pump according to claim 12, wherein a power unit houses the power housing, and the power unit is separate from the in-bra breast pump unit.

15. The breast pump according to claim 12, wherein the power source is a battery.

16. The breast pump according to claim 12, the power path being between the aggregate and the power source is a physical line.

17. The breast pump according to claim 12, the power path between the aggregate and the power source being wireless.

18. The breast pump according to claim 12, including one or more of a wireless data, and energy connection, between the in-bra breast pump unit and the power source.

19. The breast pump according to claim 18, wherein the wireless energy connection is provided via power transfer modulated by control signals.

20. The breast pump according to claim 12, wherein the power housing is provided with a user interface.

21. The breast pump according to claim 20, the user interface comprising at least one optical indicator for indicating an operational condition of the power source.

22. The breast pump according to claim 13, wherein the housing is provided with a user interface.

23. A breast pump set comprising a first in-bra breast pump unit adapted to at least in part fit inside a bra and a second in-bra breast pump unit adapted to at least in part fit inside a bra, wherein the first and the second in-bra breast pump units each include a breast shield adapted to at least in part receive a breast of a lactating user, and a milk container providing a reservoir for milk expressed from said breast, wherein the breast pump set further comprises a power unit, wherein said power unit houses a power source and said power unit is separate from the in-bra breast pump units.

24. The breast pump set according to claim 23, wherein the breast shield of the first in-bra breast pump unit and the breast shield of the second in-bra breast pump unit and/or said milk container of the first in-bra breast pump unit and the milk container of the second in-bra breast pump unit are identically shaped.

25. The breast pump according to claim 17, the power path being provided via power transfer modulated by control signals.
